(19) United States
(12) Patent Application Publication
(10) Pub. No.: US 2008/0161594 A1
(43) Pub. Date: Jul. 3, 2008

(54) Method for fabricating nanoparticles containing fenofibrate

(75) Inventors: John Jianghann Lin, Keelung City (TW); Chu Chun Hsueh, Taipei City (TW); Pei Kan, Hsinchu City (TW); Po-Fu Chou, Taoyuan County (TW); En-Wei Chang, Taichung County (TW); Wei-Liang Hsu, Taipei City (TW)

Correspondence Address: QUINTERO LAW OFFICE, PC, 2210 MAIN STREET, SUITE 200, SANTA MONICA, CA, 90405, US

(73) Assignee: INDUSTRIAL TECHNOLOGY RESEARCH INSTITUTE, HSINCHU (TW)

(21) Appl. No.: 11/618,053

(22) Filed: Dec. 29, 2006

(51) Int. Cl. C07C 69/00; B31B 1/60

(52) U.S. Cl. 560/129; 156/1

(57) ABSTRACT

A method employs the addition of additive to increase the solubility of active ingredients in solution. Furthermore, a nanoparticle apparatus that uses inkjet dispenser is utilized to fabricate nanoparticles. The method comprises: (a) mixing a fenofibrate substance, an organic solvent and a solubility enhancing additive to form a saturated solution; and (b) spray-drying the saturated solution to form the nanoparticles containing fenofibrate, wherein the solubility enhancing additive comprises a surfactant or an excipient.

### CROSS REFERENCE TO RELATED APPLICATIONS

This application is related to a co-pending U.S. application Ser. No. 11/562,958, filed on Nov. 22, 2006, the entire disclosure of which is incorporated herein by reference.

### BACKGROUND OF THE INVENTION

1. Field of the Invention

The invention relates to a method for fabricating nanoparticles containing fenofibrate.

2. Description of the Related Art

Nanotechnology is widely used in various fields such as biochemistry, medicine and chemical engineering. Regarding to medicine transfer in the biomedical field, for example, nanonization of medicines can effectively increase the total particle surface area of medicines, thus accelerating absorption rate of medicines and bioavailability. The key point of therapy using medicines is whether the medicines can be essentially (or completely) absorbed, thus particle dimensions and uniformity may directly influence the therapeutic effect.

Present nanonization of medicines may comprise physical and chemical methods. Physical methods include, for example, electrospray, ultrasound, spray drying, superior fluid, and cryogenic technology. For example, U.S. Pat. No. 6,368,620 discloses a process for preparing a nanocrystal or nanoparticle fibrate composition. U.S. Pat. No. 6,682,761 disclose a process for the preparation of small particles containing a poorly water soluble drug. U.S. Pat. No. 6,696,084 discloses a process for the preparation of small particles or microparticles containing fenofibrate and a phospholipid surface stabilizing substance. Most technologies have a common issue i.e. uneven distribution of particle diameters, which can be solved by subsequent filtering, however, manufacturing process complexity, and cost also increases. Accordingly, processes suitable for large-scale production capable of obtaining nanoparticles (such as nanoparticles containing fenofibrate) with uniform diameter are desirable.

### BRIEF SUMMARY OF THE INVENTION

One embodiment of the invention discloses a method for fabricating nanoparticles containing fenofibrate, comprising: (a) mixing a hydrophobic substance, an organic solvent and a solubility enhancing additive to form a saturated solution; and (b) spray-drying the saturated solution to form the nanoparticles containing the hydrophobic substance, wherein the solubility enhancing additive comprises a surfactant or an excipient.

Another embodiment of the invention discloses a method for fabricating nanoparticles containing fenofibrate and that the hydrophobic substance comprises fenofibrate.

Another embodiment of the invention discloses a method for fabricating nanoparticles containing fenofibrate and that the solvent comprises an organic solvent.

Another embodiment of the invention discloses a method for fabricating nanoparticles containing fenofibrate and that the organic solvent comprises alcohol.

Another embodiment of the invention discloses a method for fabricating nanoparticles containing fenofibrate and that the additive comprises a surfactant or excipient.

The solubility and of active ingredients (for example, fenofibrate) in solution can be increased by means of utilizing the solubility enhancing additive.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows one embodiment of a nanoparticle fabrication method.

FIG. 2 shows one embodiment of a system for fabricating nanoparticles.

FIG. 3 shows one embodiment of the particle size distribution.

FIG. 4 shows one embodiment of the particle size distribution.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

Embodiments of the invention provide methods for fabricating nanoparticles from a supersaturated liquid solution with a substance to be transformed into nanoscale. The liquid solution is preferably composed of a solvent, a solubility enhancing additive, and the substance to be transformed into nanoscale dissolved therein. The solvent, for example, can be alcohol (also ethanol). However, other solvents, or mixtures of solvents, which can dissolve the substance and are miscible with the anti-solvent selected in the nanoparticle formation device are also suitable. An example of a solubility enhancing additive is a surfactant (i.e. Brij 76 (purchased from Sigma-Aldrich, St. Louis, Mo.)); nonetheless, other additives that are able to increase the intrinsic solubility of the substance in the solvent are also included. In addition, substances suitable to be transformed into nanoscale include bioactive material, polymer material, biomaterial, chemical material or mixtures thereof. Note that the substances are active agents in the solvent. Furthermore, the additive is a stabilizer or an excipient.

Nanoparticle fabrication, the process of which a substance to be transformed into nanoparticle, is done via fabrication apparatus and process described later.

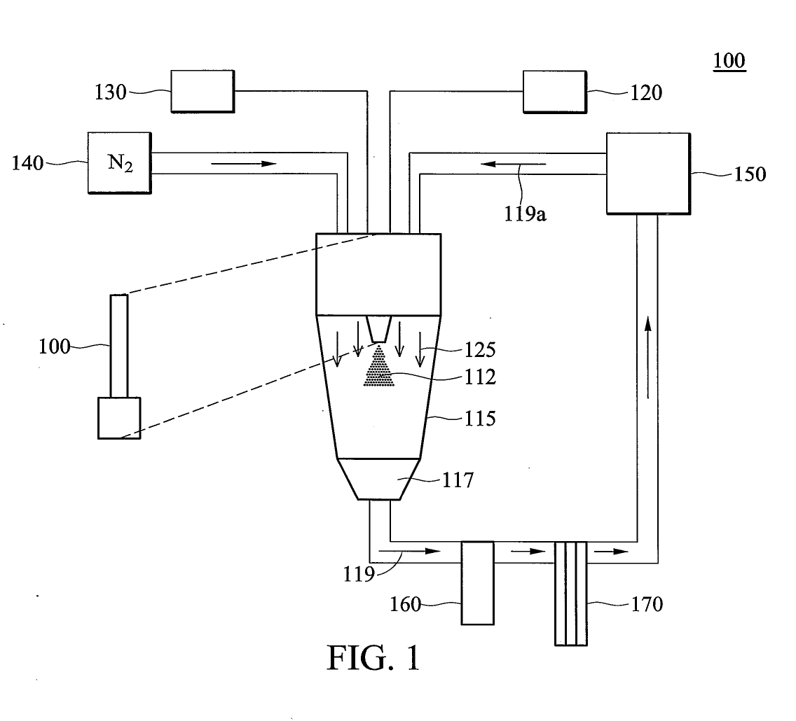

FIG. 1 shows an embodiment of a nanoparticle fabrication method. As shown in FIG. 1, the system 100 includes a micro droplet sprayer 110, a drying chamber 115, a liquid supplier and a pressure controller 120 of the micro droplet sprayer 110, a device (e.g. a controller or a control system) 130 of the micro droplet sprayer 110, a nitrogen supplier 140 of the system 100, an inner loop 150 of the system 100, a particle collector 160 and a particle filter 170.

The micro droplet sprayer 110, for example, can be an inkjet sprayer including a liquid tank (not shown), a channel (not shown), an actuator (not shown), and orifices (not shown). The actuator drives several orifices to spray the solution, thus micro droplets 112 are generated. The actuator can be a thermal bubble actuator or a piezoelectric actuator. In embodiments of the invention, the solution such as a medicine solution employing alcohol as a solvent is poured into the micro droplet sprayer 110. The drying chamber 115 is used to collect and dry the droplets 112, and it can be a thermal dryer or a hot air generator. The liquid supplier and pressure controller 120 are capable of supplying liquid steadily and controlling the pressure required by micro droplet sprayer 110, thus avoiding the pressure change rendered by the volume change of solution during operation. Driving forces of the pressure controller 120 comprise mechanical forces, atmosphere difference or potential difference. The device (e.g. a controller or a control system) 130 can provide the micro droplet sprayer 110 with various energy pulses or other parameters for spraying liquid. The nitrogen suppliers 140 are provided for keeping oxygen concentration to less than a specific value by steadily providing the system with nitrogen because the system 100 utilizes an organic solvent as solvent of the medicinal solution to be sprayed and is operated under high temperature that may cause an explosion. The inner loop 150 can recycle the nitrogen (the heated nitrogen can be used as hot air) and condense organic solvent for collection. The particle collector 160 and particle filter 170 can prevent particles from escaping into the air.

The liquid supplier and pressure controller 120 inject the medicine solution into the micro droplet sprayer 110. In addition, The micro droplet sprayer 110 is driven by the device (e.g. a controller or a control system) 130 to spray the medicine solution, thus micro droplets 112 are formed in the drying chamber 115. The nitrogen supplier 140 simultaneously injects nitrogen into the drying chamber 115, generating hot air 125 and drying the micro droplets 112 released from the micro droplet sprayer 110. As a result, nanoparticles (i.e. the dried micro droplets 112) are obtained. The nanoparticles then settle to the bottom 117 of drying chamber 115 for collection by the particle collector 160 following the direction of arrow 119. The nanoparticles, remaining in the nitrogen, however, are trapped by the particle filter 170. The used nitrogen is then recycled by means of the inner loop 150 and enters the drying chamber 115 again. In embodiments of the invention, an auxiliary element (not shown) for controlling spray directions of the droplets 112 is provided, thus avoiding turbulence or collision therebetween during operation of micro droplet sprayer 110. In addition, the auxiliary element is arranged in a front end of the micro droplet sprayer and the shape of the auxiliary element is cylindrical or conical.

As shown in FIG. 1, the processes and parameters for the system 100 are described as the following. First, the drying chamber 115 is filled with nitrogen and heated to a desired temperature e.g., 100° C. When the system reaches a steady state, the micro droplet sprayer 110 is driven to steadily spray the medicinal solution, forming the droplets 112. In addition, the medicinal solution includes alcohol as solvent and the spray frequency is 0.3 kHz. Subsequently, nanoparticles are rapidly obtained due to the small size of the droplets 112 are tiny and sprayed into a high temperature ambient. Specifically, the described nanoparticles have uniform diameters due to recipes of the solutions. Finally, nanoparticles are collected by the particle collector 160.

In the following five embodiments, a medicine solution containing fenofibrate is employed in the system 100 shown in FIG. 1, fabricating nanoparticles containing fenofibrate. The same or similar apparatus and processes are omitted in each embodiment.

### First Embodiment

The solubility of fenofibrate (substance) in ethanol was increased from value of 2.5% (w/v) with an excipient such as poly vinyl pyrrolidone (PVP) at substance to excipient ratio of 1:1 to 10% (w/v) with an excipient such as Brij 76 (purchased from Sigma-Aldrich, St. Louis, Mo.) at substance to excipient ratio of 1:1. Precipitation of substance was observed overnight suggesting supersaturation phenomenon.

### Second Embodiment

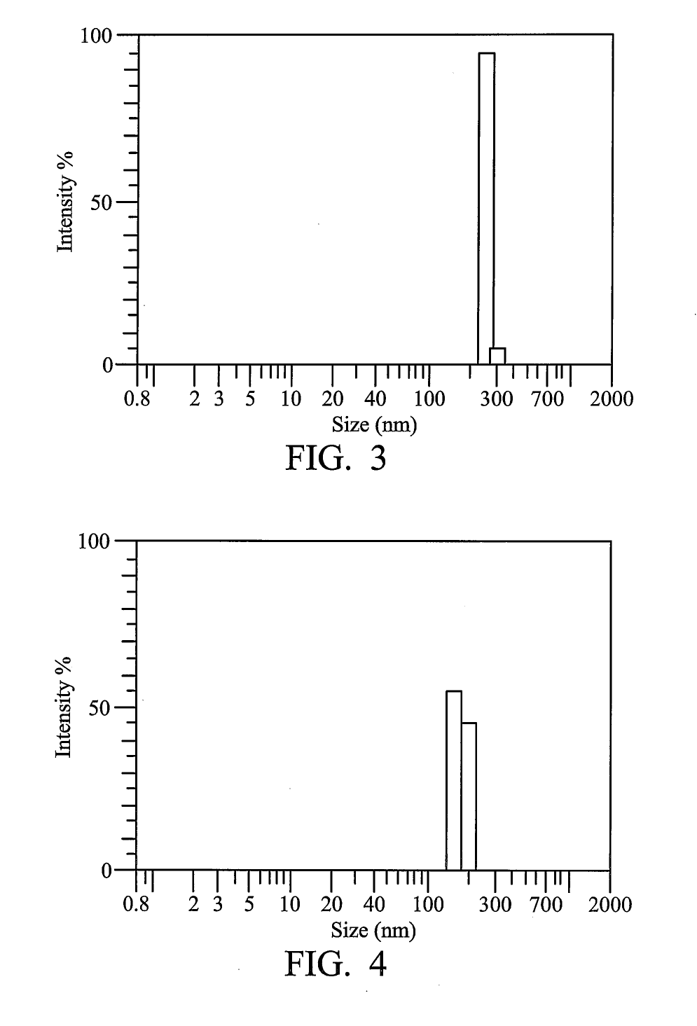

The solubility of fenofibrate (substance) in ethanol was increased from value of 2.5% (w/v) with an excipient such as poly vinyl pyrrolidone (PVP) at substance to excipient ratio of 1:1 to 10% (w/v) with an excipient such as Brij 76 (purchased from Sigma-Aldrich, St. Louis, Mo.) at substance to excipient ratio of 1:2. As shown in FIG. 3, the particle size of the substance (fenofibrate) produced by the nanonization apparatus (inkjet spray-dryer) is 287.3 nm+/−102.9 μm. The distribution range of the particles is in the nanoscale range of 251.2 nm (95.0%)−316.2 nm (4.6%); thus, illustrates that the particles produced are uniformly distributed. The percentage indicated in the parentheses is intensity percentage of the particle measured using dynamic light scattering (DLS) technique.

### Third Embodiment

The solubility of fenofibrate (substance) in ethanol was increased from value of 2.5% (w/v) with an excipient such as poly vinyl pyrrolidone (PVP) at substance to excipient ratio of 1:1 to 10% (w/v) with an excipient such as D-alpha-tocopheryl polyethylene glycol 1000 succinate (vitamin E TPGS), purchased from Eastman Chemical Company, Kingsport, Tenn.) at substance to excipient ratio of 1:1. Precipitation of substance was observed overnight suggesting supersaturation phenomenon.

### Fourth Embodiment

The solubility of fenofibrate (substance) in ethanol was increased from value of 2.5% (w/v) with an excipient such as poly vinyl pyrrolidone (PVP) at substance to excipient ratio of 1:1 to 10% (w/v) with an excipient such as D-alpha-tocopheryl polyethylene glycol 1000 succinate (vitamin E TPGS), purchased from Eastman Chemical Company, Kingsport, Tenn. at substance to excipient ratio of 1:2. Precipitation of substance was observed overnight suggesting supersaturation phenomenon. As shown in FIG. 4, the particle size of the substance (fenofibrate) produced by the nanonization apparatus (inkjet spray-dryer) is 192.8 nm+/−47.2 nm. The distribution range of the particles is in the nanoscale range of 158.5 nm (55.8%)−199.5 nm (44.2%); thus, illustrates that the particles produced are uniformly distributed. The percentage indicated in the parentheses is intensity percentage of the particle measured using dynamic light scattering (DLS) technique.

### Fifth Embodiment

The solubility of fenofibrate (substance) in ethanol was increased from value of 2.5% (w/v) with an excipient such as poly vinyl pyrrolidone (PVP) at substance to excipient ratio of 1:1 to 10% (w/v) with an excipient such as Solutol HS15 mainly including poly-oxyethylene esters of 12-hydroxystearic acid (manufactured by BASF, Florham Park, N.J.) at substance to excipient ratio of 1:1.

The solubility of fenofibrate (substance) in ethanol was increased from value of 2.5% (w/v) with an excipient such as poly vinyl pyrrolidone (PVP) at substance to excipient ratio of 1:1 to 10% (w/v) with an excipient such as Arlacel 83 mainly including sorbitan sesquioleate (manufactured by Stobec, Quebec, Canada) at substance to excipient ratio of 1:1.

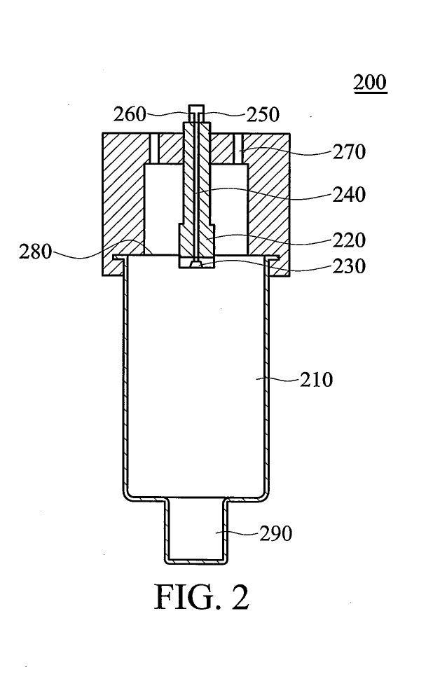

FIG. 2 shows one embodiment of a system for fabricating nanoparticles. As shown in FIG. 2, the system 200 e.g. a hot air drying system dries the droplets using hot air, thus, nanoparticles are formed. The system 200 includes a drying chamber 210, micro droplet sprayer 220, orifices 230 of micro droplet sprayer, pipes 240, nitrogen entrance 250, water (from circulation chamber) entrance 260, hot air entrance 270, hot air exit 280, and the bottom 290 of drying chamber 210. First to fifth embodiments are also suitable to the system 200.

As described, the invention fabricates nanoparticles with uniform diameters by integrating injection printing techniques into subsequent drying and formation processes. In addition, the system is further equipped with the auxiliary element for controlling spray directions of the droplets and particle collector for collecting dried nanoparticles. Compared to the related art, the invention has advantages such as low cost, fine droplets, uniform droplet diameters, and simple apparatus and processes. Specifically, the nanoparticles fabricated by the invention have uniform particle diameters, thus, they can be used to manufacture medicines enhancing absorption and solubility in the blood. The invention aids in improving the therapeutic effect of medicines.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for fabricating nanoparticles containing fenofibrate, comprising:
(a) mixing fenofibrate, an organic solvent and a solubility enhancing additive to form a saturated solution; and
(b) spray-drying the saturated solution to form the nanoparticles containing fenofibrate using a system.

2. The method as claimed in claim 1, wherein the solubility enhancing additive is a surfactant or an excipient.

3. The method as claimed in claim 1, wherein the solubility enhancing additive is selected from the group consisting of Brij 76, TPGS, Solutol HS15 and Arlacel 83.

4. The method as claimed in claim 1, wherein the organic solvent comprises alcohol.

5. The method as claimed in claim 1, wherein a ratio of fenofibrate to the solubility enhancing additive ranges between 0.1 and 10.

6. The method as claimed in claim 1, wherein a ratio of fenofibrate to the solubility enhancing additive ranges between 0.2 and 5.

7. The method as claimed in claim 1, wherein a ratio of fenofibrate to the solubility enhancing additive ranges between 0.33 and 3.

8. The method as claimed in claim 1, wherein a ratio of fenofibrate to the solubility enhancing additive ranges between 0.5 and 2.

9. The method as claimed in claim 1, wherein a solubility of the fenofibrate is larger than 3%.

10. The method as claimed in claim 1, wherein a solubility of the fenofibrate is larger than 5%.

11. The method as claimed in claim 1, wherein a solubility of the fenofibrate is larger than 8%.

12. The method as claimed in claim 1, wherein a size of the nanoparticles ranges between 1 and 1000 nm.

13. The method as claimed in claim 1, wherein a size of the nanoparticles ranges between 50 and 500 nm.

14. The method as claimed in claim 1, wherein a size of the nanoparticles ranges between 100 and 350 nm.

15. The method as claimed in claim 1, wherein the system comprises:
a micro droplet sprayer, wherein the micro droplet sprayer is an inkjet sprayer utilized for generation of micro droplets;
a device employed to provide the micro droplet sprayer with energy, forcing the droplets out; and
a drying chamber, wherein the droplets are dried therein.

16. Nanoparticles containing fenofibrate prepared according to the method of claim 1.

17. The nanoparticles as claimed in claim 16, wherein a size of the nanoparticles ranges between 1 and 1000 nm.

18. The nanoparticles as claimed in claim 16, wherein a size of the nanoparticles ranges between 50 and 500 nm.

19. The nanoparticles as claimed in claim 16, wherein a size of the nanoparticles ranges between 100 and 350 nm.
